(19) United States
(12) Patent Application Publication
(10) Pub. No.: US 2026/0053363 A1
(43) Pub. Date: Feb. 26, 2026

(54) MULTI-CHANNEL PULSE HIGH-VOLTAGE PARAMETER-CONTROLLABLE SHOCK WAVE LITHOTRIPSY BALLOON IMAGING SYSTEM AND CATHETER THEREOF

(71) Applicant: NANJING FORSSMANN MEDICAL TECHNOLOGY CO., LTD., Nanjing, Jiangsu (CN)

(72) Inventors: Guosheng FU, Nanjing, Jiangsu (CN); Chongying JIN, Nanjing, Jiangsu (CN); Chengqing PENG, Nanjing, Jiangsu (CN); Guojia WU, Nanjing, Jiangsu (CN); Hao KUANG, Nanjing, Jiangsu (CN); Yanle DENG, Nanjing, Jiangsu (CN); Qiuyang GU, Nanjing, Jiangsu (CN); Kai SHEN, Nanjing, Jiangsu (CN); Yan ZHANG, Nanjing, Jiangsu (CN); Tong WAN, Nanjing, Jiangsu (CN); Feng BAO, Nanjing, Jiangsu (CN)

(21) Appl. No.: 19/140,778

(22) PCT Filed: Dec. 6, 2023
(86) PCT No.: PCT/CN2023/136637

§ 371 Date: Jun. 18, 2025

(30) Foreign Application Priority Data
Dec. 29, 2022 (CN) 202211428262.9

(51) Int. Cl. A61B 5/00; A61B 17/22

(52) U.S. Cl. CPC A61B 5/0036; A61B 5/0066; A61B 5/0084; A61B 5/6853; A61B 17/22022; A61B 2017/22025

(57) ABSTRACT

Provided are a multi-channel pulse high-voltage parameter-controllable shock wave lithotripsy balloon imaging system and a catheter thereof. An intravascular imaging catheter is arranged in a shock wave lithotripsy balloon, and a plurality of high-coverage electrode pairs capable of releasing shock wave energy are arranged in a working area of the shock wave lithotripsy balloon.

### TECHNICAL FIELD

The present disclosure belongs to the technical field of medical device, and relates to a multi-channel pulsed high-voltage parameter-controllable shock wave lithotripsy balloon imaging system and a catheter thereof.

### BACKGROUND ART

In recent years, with the progression of global population aging and the enhancement of quality of life, not only among aged population, but also in some young and middle-aged populations, incidence rates of various vascular diseases are continuously rising. Atherosclerosis is an arterial stenosis and calcification disease caused by plaque accumulation. Plaques are composed of fibrous tissues, calcium, etc. The aggregated calcified plaques hinder normal flow of blood in blood vessels and reduce supply of oxygen and nutrients to the body.

Currently, clinical practice commonly employs the conventional balloon catheter interventional therapy, to dilate calcified lesions in blood vessels through balloon catheter vascular dilation techniques. However, under pressure release of a dilation balloon, and in the absence of specific observation of intravascular calcified lesion distribution, the dilation balloon may fail to achieve expected therapeutic efficacy, and even a vascular wall may be damaged due to pressure during balloon dilation.

Intravascular shock wave therapy utilizes the principle of liquid-phase discharge of an electrode pair in a medium within a balloon, and shock waves formed in a discharge process act on the balloon and are transmitted to calcified tissues, while the balloon is tightly attached around the calcified tissues under a certain air pressure condition. As mechanical waves, the shock waves achieve acoustic impedance matching with the calcified tissues, and shock wave energy propagates through the balloon to reach the calcified tissues, and causes calcified plaques to crack and break, and the balloon can undergo further inflation to dilate the blood vessel, thereby achieving an objective of vascular remodeling.

Published patent CN104958065A describes an intravascular tomographic imaging catheter, also known as an OCT imaging catheter, where by utilizing the basic principle of a weak-coherent light interferometer, an optical fiber lens is built in the catheter, so that an image of a blood vessel can be observed in real time under high-speed rotation. Moreover, calcified plaques have very strong reflectivity to near-infrared weak-coherent light, and it is easier to identify locations of calcified plaques. The present disclosure, based on the above OCT imaging catheter, integrates processes such as the balloon and electrodes in a shock wave treatment catheter, and realizes OCT image calcification assessment under shock wave treatment.

Published patent CN114903559A describes an integrated optical coherence tomography shock wave balloon catheter and a system thereof. The patent mentions an integrated system of OCT imaging and shock wave treatment. The system neither mentions human coronary, peripheral and valvular high-voltage module application, nor mentions an AI intelligent calcification assessment mechanism of an OCT system and a high-voltage module. The present disclosure is advantageous over the integrated OCT imaging system mentioned in the above.

### SUMMARY

The present disclosure provides a multi-channel pulsed high-voltage parameter-controllable shock wave lithotripsy balloon imaging system and a catheter thereof, belonging to the technical field of medical device. The multi-channel pulsed high-voltage parameter-controllable shock wave lithotripsy balloon imaging system and the catheter thereof in the present disclosure belong to integrated innovation of vascular remodeling technique, shock wave therapeutic technique and intravascular tomographic imaging technique. In the present disclosure, a shock wave lithotripsy balloon is provided with an intravascular imaging catheter therein, and a plurality of high-coverage electrode pairs that can release shock wave energy are provided in an operation area of the shock wave lithotripsy balloon.

A multi-channel pulsed high-voltage parameter-controllable shock wave lithotripsy balloon imaging system and a catheter thereof in the present disclosure include: a balloon imaging catheter, an intravascular tomographic imaging system, and a multi-channel pulsed high-voltage parameter-adjustable module. The balloon imaging catheter is obtained by laser-welding a balloon on, for example, an imaging window of a TY series catheter from NANJING FORSSMANN MEDICAL TECHNOLOGY CO., LTD., for example, on an imaging window of a disposable intravascular imaging catheter (TY-1), where the balloon is provided therein with a developing ring and multiple sets of electrode pairs, and electrodes in the electrode pairs are connected to a connector at a distal end of the catheter via a lead wire. The TY series catheter may be, for example, an intravascular tomographic imaging catheter from NANJING FORSSMANN MEDICAL TECHNOLOGY CO., LTD., and the catheter is provided with an optical fiber therein, and is connected to an intravascular tomographic imaging system through a connector. The multi-channel pulsed high-voltage parameter-adjustable system has modules with adjustable high voltage, pulse width, repetition frequency and pulse burst count. With the technical solutions in the present disclosure and routes thereof, the intravascular tomographic imaging system is integrated with a shock wave treatment function for vascular calcified tissues, such that surgeons perform effective treatment and assessment “before, in and after operation”, and through integration with AI calcification assessment software of the intravascular tomographic imaging system, can effectively identify a calcification category and perform accurate shock wave treatment for deep calcification, thereby shortening a treatment time, reducing a learning curve of surgeons, and improving therapeutic efficacy of surgery.

The first aspect of the present disclosure provides a multi-channel pulsed high-voltage parameter-controllable shock wave lithotripsy balloon imaging catheter, including an imaging catheter body, an operation balloon, a first operation electrode pair, a second operation electrode pair and a sheath. The imaging catheter body includes an imaging window. The operation balloon includes an operation area. The operation balloon is provided in the imaging window. The operation balloon is provided with a first operation balloon pin and a second operation balloon pin. The first operation balloon pin and the imaging window form an enclosed space, and the second operation balloon pin and a circumferential wall of the sheath form an enclosed space. The first operation electrode pair and the second operation electrode pair are provided in the operation area of the operation balloon and on the imaging window.

In a preferred example, the first operation electrode pair includes: a first operation electrode, a second operation electrode and a first insulating layer; the second operation electrode pair includes: a third electrode, a fourth electrode and a second insulating layer, where the first electrode is provided on the imaging window, the first insulating layer is provided on the first electrode, and the second electrode is provided on the first insulating layer.

In another preferred example, the first insulating layer is provided with a first energy release window, and the second electrode is provided with a second energy release window, where the second energy release window is slightly larger than the first energy release window, and the first energy release window and the second energy release window are coaxial and co-directional; and the third electrode is provided with a third energy release window, and the fourth electrode is provided with a fourth energy release window, where the fourth energy release window is slightly larger than the third energy release window, and the third energy release window and the fourth energy release window are coaxial and co-directional.

In another preferred example, the multi-channel pulsed high-voltage parameter-controllable shock wave lithotripsy balloon imaging catheter includes: a first lead wire and a second lead wire, where the first lead wire is connector to the first electrode and the third electrode, and the second lead wire is connected to the second electrode and the fourth electrode.

In another preferred example, the multi-channel pulsed high-voltage parameter-controllable shock wave lithotripsy balloon imaging catheter includes: an electrical connector, and the first lead wire and the second lead wire extend along a direction from the operation balloon to the electrical connector and are connected to the electrical connector.

In another preferred example, the multi-channel pulsed high-voltage parameter-controllable shock wave lithotripsy balloon imaging catheter includes: an optical lens, an optical fiber, a traction wire and an optical lens base, where the optical lens is connected to the optical fiber, and the traction wire is connected to the optical fiber and the optical lens base.

In another preferred example, the imaging catheter body is provided with a first rapid exchange port, and the imaging window is provided with a second rapid exchange port.

In another preferred example, the multi-channel pulsed high-voltage parameter-controllable shock wave lithotripsy balloon imaging catheter includes: a first developing ring, a second developing ring and a third developing ring, where the first developing ring is provided adjacent to the second rapid exchange port, the second developing ring and the third developing ring are provided at two ends inside the operation balloon, respectively, the second developing ring is provided adjacent to the first operation electrode pair, and the third developing ring is provided adjacent to the second operation electrode pair.

In another preferred example, the first operation electrode, the second operation electrode, the third operation electrode, and the fourth operation electrode are made of a material such as tungsten, platinum-iridium alloy, and stainless steel alloy; shapes of the first electrode and the third electrode include an annular shape, a disk shape and a square sheet shape; and shapes of the second electrode and the fourth electrode include an annular shape, a semi-annular shape and an annular shape provided with a wire slot.

The second aspect of the present disclosure provides a multi-channel pulsed high-voltage parameter-controllable shock wave lithotripsy balloon imaging system, including a high-voltage connector, a flange connector, a Luer taper, an intravascular tomographic imaging system, a multi-channel pulsed high-voltage parameter-adjustable module and the multi-channel pulsed high-voltage parameter-controllable shock wave lithotripsy balloon imaging catheter provided in the first aspect of the present disclosure. The intravascular tomographic imaging system is configured to assess intravascular imaging and/or vascular calcification. The multi-channel pulsed high-voltage parameter-adjustable module includes a high-voltage adjustable module, a pulse-width adjustable module, a repetition-frequency adjustable module and a pulse-burst-count adjustable module. The multi-channel pulsed high-voltage parameter-controllable shock wave lithotripsy balloon imaging system is connected to the multi-channel pulsed high-voltage parameter-adjustable module and the intravascular tomographic imaging system via the high-voltage connector and the flange connector. The Luer taper is configured to inject a shock wave transmission medium into the operation balloon, where the transmission medium includes: saline and/or a contrast agent.

It should be understood that within the scope of the present disclosure, various technical features of the present disclosure in the above and various technical features specifically described below (e.g., in the examples) can be combined with each other, so as to form new or preferred technical solutions, which are not repeated herein one by one due to limited length.

The above description is merely a summary of the technical solutions of the present disclosure. Implementation can be carried out according to contents of the specification in order to be capable of understanding technical means of the present disclosure more clearly. In order to make the above objectives, features and advantages of the present disclosure more obvious and more understandable, preferable embodiments are particularly illustrated in the following to give detailed description in conjunction with the drawings.

### BRIEF DESCRIPTION OF DRAWINGS

FIG. 1 is a schematic view of a connection relationship between a multi-channel pulsed high-voltage parameter-controllable shock wave lithotripsy balloon imaging system and a catheter thereof in the present disclosure;

FIG. 2 is a partial front view of a main operation area of a multi-channel pulsed high-voltage parameter-controllable shock wave lithotripsy balloon imaging system and a catheter thereof in embodiments of the present disclosure;

FIG. 3 is a partial top view of a main operation area of a multi-channel pulsed high-voltage parameter-controllable shock wave lithotripsy balloon imaging system and a catheter thereof in embodiments of the present disclosure;

FIG. 4 is a partial sectional view of a main operation area of a multi-channel pulsed high-voltage parameter-controllable shock wave lithotripsy balloon imaging system and a catheter thereof in embodiments of the present disclosure; and FIG. 5 is a schematic view of a multi-channel pulsed high-voltage parameter-controllable shock wave lithotripsy balloon imaging system in embodiments of the present disclosure.

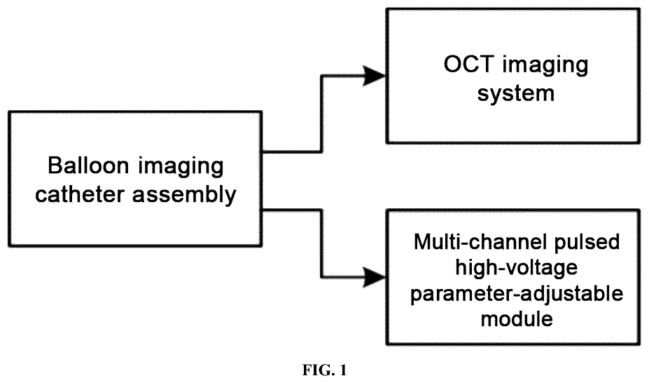

### Illustration of Reference Signs

01A—first operation electrode pair; 01B—second operation electrode pair;
02—operation balloon; 03A—imaging catheter;
03B—catheter imaging window; 04, 05—rapid exchange port;
06A—first lead wire; 06B—second lead wire;
07A—first developing ring; 07B—second developing ring; 07C—third developing ring;
08A—first energy release window; 08B—third energy release window;
09A—second energy release window; 09B—fourth energy release window;
10—sheath; 20—optical lens; 30—traction wire; 40—optical fiber;
50A—first operation electrode; 50B—third operation electrode;
60A—first insulating layer; 60B—second insulating layer;
70A—second operation electrode; 70B—fourth operation electrode;
80A, 80B—operation balloon pin; 90A—electrical connector;
90B—catheter connector; 100—optical lens base;
101—push handle; 102—injection port.

### DETAILED DESCRIPTION OF EMBODIMENTS

Objectives, implementation technical solutions and use advantages of embodiments of a multi-channel pulsed high-voltage parameter-controllable shock wave lithotripsy balloon imaging system and a catheter thereof in the present disclosure will be fully described below in conjunction with drawings in the embodiments of the multi-channel pulsed high-voltage parameter-controllable shock wave lithotripsy balloon imaging system and the catheter thereof in the present disclosure.

The present disclosure provides a multi-channel pulsed high-voltage parameter-controllable shock wave lithotripsy balloon imaging system and a catheter thereof. Embodiments of the present disclosure mainly aim at addressing a current problem of inability to perform real-time treatment on observed intravascular calcified lesions while conducting real-time, specific observation of the intravascular calcified lesions during interventional therapy for intravascular calcified lesions.

A multi-channel pulsed high-voltage parameter-controllable shock wave lithotripsy balloon imaging system and the catheter thereof presented in embodiments of the present disclosure can perform real-time shock wave treatment on calcified lesions within a presented scanned area while presenting intravascular calcified lesions through imaging catheter scanning, and can timely and effectively treat the intravascular calcified lesions, enhance a patency rate of blood vessels with calcified lesions, and improve a blood flow rate in vessels.

A multi-channel pulsed high-voltage parameter-controllable shock wave lithotripsy balloon imaging catheter provided in the present disclosure is characterized by including a balloon imaging catheter, a high-voltage connector, a flange connector, and a Luer taper. The balloon imaging catheter is obtained by laser-welding a balloon on, for example, an imaging window of a TY series catheter from NANJING FORSSMANN MEDICAL TECHNOLOGY CO., LTD., for example, on an imaging window of a disposable intravascular imaging catheter (TY-1), where the balloon is provided therein with a developing ring and multiple sets of electrode pairs, and electrodes in the electrode pairs are connected to a high-voltage connector at a distal end of the catheter via a lead wire, and connected to a high-voltage adjustable module via the high-voltage connector. The TY series catheter may be, for example, an intravascular tomographic imaging catheter from NANJING FORSSMANN MEDICAL TECHNOLOGY CO., LTD., and the catheter is provided with an optical fiber therein, and is connected to the intravascular tomographic imaging system through the flange connector. The Luer taper is configured to inject a shock wave transmission medium, typically saline and a contrast agent in a defined ratio, into the balloon.

A multi-channel pulsed high-voltage parameter-controllable shock wave lithotripsy balloon imaging system related in embodiments of the present disclosure includes: an imaging catheter 03A, an operation balloon 02, a first operation electrode pair 01A, a second operation electrode pair 01B, a first lead wire 06A, a second lead wire 06B, and a sheath 10.

The drawings in the embodiments of the present disclosure specifically show a partial view and an internal sectional view of an operation area of the multi-channel pulsed high-voltage parameter-controllable shock wave lithotripsy balloon imaging system and the catheter thereof in the present disclosure, and a schematic view of lead wires of operation electrodes of the catheter extending to an external electrical connector.

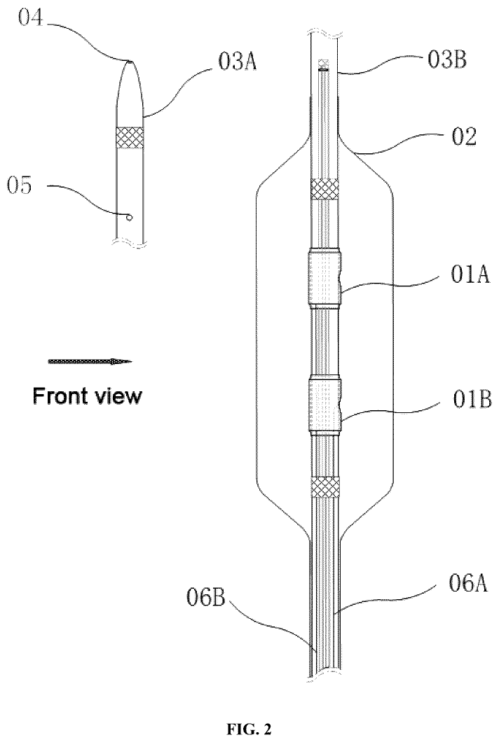

FIG. 2 is a partial front view of a main operation area of a multi-channel pulsed high-voltage parameter-controllable shock wave lithotripsy balloon imaging system and a catheter thereof in an embodiment of the present disclosure. In the drawing of the present embodiment, a first catheter rapid exchange port 04, a second catheter rapid exchange port 05, an imaging catheter 03A, a catheter imaging window 03B, an operation balloon 02, a first operation electrode pair 01A, a second operation electrode pair 01B, a first lead wire 06A and a second lead wire 06B are marked.

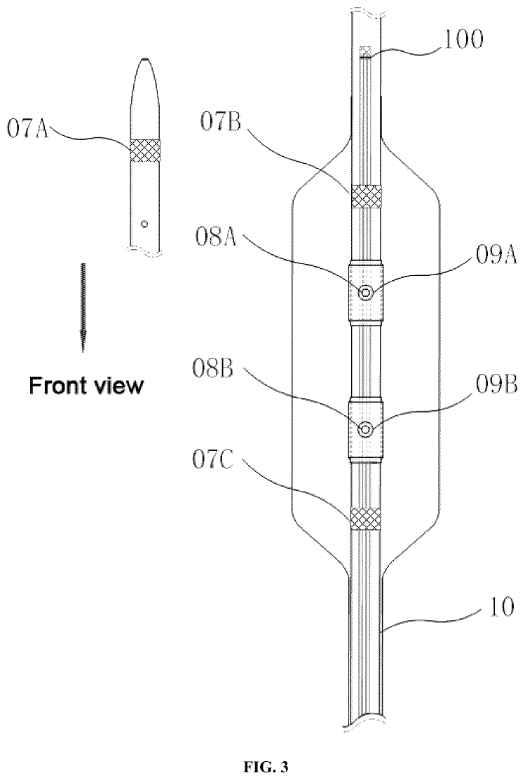

FIG. 3 is a partial top view of a main operation area of a multi-channel pulsed high-voltage parameter-controllable shock wave lithotripsy balloon imaging system and a catheter thereof in an embodiment of the present disclosure. In the drawing of the present embodiment, a first developing ring 07A, a second developing ring 07B, a third developing ring 07C, a first energy release window 08A, a second energy release window 08B, a third energy release window 09A, a fourth energy release window 09B and an optical lens base 100 are marked.

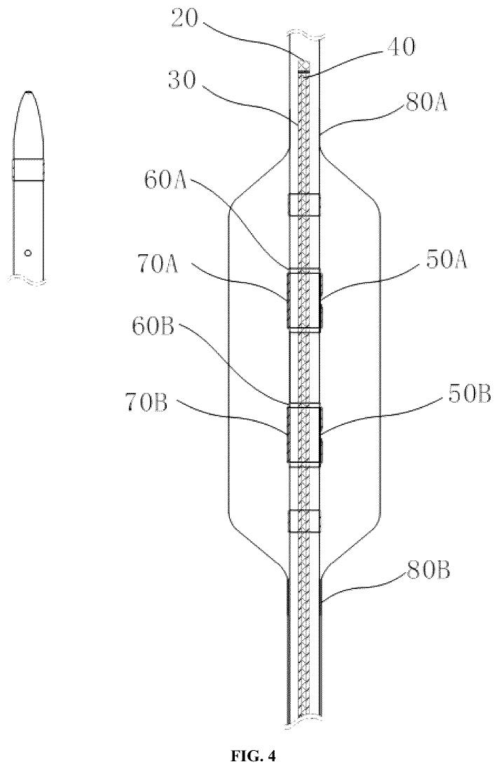

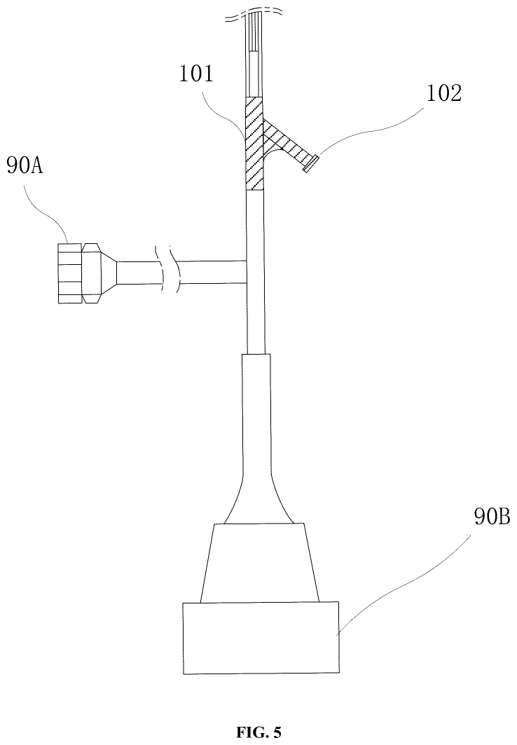

FIG. 4 is a partial sectional view of a main operation area of a multi-channel pulsed high-voltage parameter-controllable shock wave lithotripsy balloon imaging system and a catheter thereof in an embodiment of the present disclosure. In the drawing of the present embodiment, an optical lens 20, an optical fiber 40, a traction wire 30, a first operation balloon pin 80A, a second operation balloon pin 80B, a first operation electrode 50A, a second operation electrode 50B, a third operation electrode 70A, a fourth operation electrode 70B, a first insulating layer 60A, and a second insulating layer 60B are marked.

In embodiments of the present disclosure, the optical lens 20 is fixed on the optical lens base 100, the optical lens 20 and the optical fiber 40 are connected into one piece, the traction wire 30 and the optical lens 20 are closely welded, and the traction wire 30 closely wraps the optical fiber 40. The optical lens 20, the optical lens base 100, the optical fiber 40, and the traction wire 30 are provided inside the catheter imaging window 03B.

In embodiments of the present disclosure, the operation balloon 02 is provided in an area of the catheter imaging window 03B, the sheath 10 is provided in an operation area of the second operation balloon pin 80B and the catheter imaging window 03B and extends to a catheter push handle 101. For the first operation balloon pin 80A and the second operation balloon pin 80B, the first operation balloon pin 80A and a circumferential wall of the catheter imaging window 03B form an enclosed space, and the second operation balloon pin 80B and a circumferential wall of the sheath 10 form an enclosed space.

In embodiments of the present disclosure, the first operation electrode pair 01A and second operation electrode pair 01B are provided in an operation area of the operation balloon 02, an operation area of the optical lens 20, and an outer wall of the catheter imaging window 03B.

In embodiments of the present disclosure, the first operation electrode pair 01A includes: the first operation electrode 50A, the second operation electrode 70A, and the first insulating layer 60A. The first insulating layer 60A is provided with the first energy release window 08A, the second operation electrode 70A is provided with the second energy release window 09A, and the second energy release window 09A is slightly larger than the first energy release window 08A. The first operation electrode 50A is provided on the outer wall of the catheter imaging window 03B, the first insulating layer 60A is provided on the first operation electrode 50A, the second operation electrode 70A is provided on the first insulating layer 60A, and the first energy release window 08A and the second energy release window 09A are coaxial and co-directional.

In embodiments of the present disclosure, the second operation electrode pair 01B includes: the third operation electrode 50B, the fourth operation electrode 70B, and the second insulating layer 60B. The second insulating layer 60B is provided with the third energy release window 08B, the fourth operation electrode 70B is provided with the fourth energy release window 09B, and the fourth energy release window 09B is slightly larger than the third energy release window 08B. The third operation electrode 50B is provided on the outer wall of the catheter imaging window 03B, the second insulating layer 60B is provided on the third operation electrode 50B, the fourth operation electrode 70B is provided on the second insulating layer 60B, and the third energy release window 08B and the fourth energy release window 09B are coaxial and co-directional.

In embodiments of the present disclosure, the first lead wire 06A is connected to the first operation electrode 50A and the third operation electrode 70A, and the second lead wire 06B is connected to the second operation electrode 50B and the fourth operation electrode 70B; and the first lead wire 06A and the second lead wire 06B extend along a direction from the imaging catheter 03A to an electrical connector 90A and are connected to the electrical connector 90A.

In embodiments of the present disclosure, the multi-channel pulsed high-voltage parameter-controllable shock wave lithotripsy balloon imaging system and the catheter thereof include, but are not limited to, two sets of operation electrode pairs, and the second and fourth operation electrodes 70A and 70B may include, but are not limited to, electrode rings, semi-electrode rings, and electrode rings provided with a wire slot made of materials such as tungsten, stainless steel, platinum-iridium alloy, and copper alloy. The first and third operation electrodes 50A and 50B may be metal rings, semi-electrode rings, metal sheets, metal lead wires, or the like made of a metal material in the first and fourth operation electrodes 50A and 70B.

In embodiments of the present disclosure, the multi-channel pulsed high-voltage parameter-controllable shock wave lithotripsy balloon imaging system and the catheter thereof are provided with a first rapid exchange port 04 and a second rapid exchange port 05 in a front section, so that the multi-channel pulsed high-voltage parameter-controllable shock wave lithotripsy balloon imaging system and the catheter thereof provided in the present disclosure, guided by a guiding catheter or a guide wire, can quickly enter an intravascular calcified lesion position. A developing agent is injected into the imaging catheter 03A. The operation balloon 02 is filled with a conductive agent, and distribution of calcium compounds in an intravascular calcified lesion area is quickly and effectively presented on the imaging system through an intravascular imaging function of the catheter, to precisely locate the calcified lesion. Then the operation electrode pairs are enabled to release shock wave energy through a pulsed high-voltage power supply control system. The shock wave energy reaches a surface of the operation balloon 02 through the conductive agent, and is uniformly distributed and applied on the located calcified lesion, thus achieving an effective therapeutic efficacy for this intravascular calcified lesion, improving the patency rate of vessels, and elevating surgical efficiency.

A multi-channel pulsed high-voltage parameter-controllable shock wave lithotripsy balloon imaging system is characterized by including an intravascular tomographic imaging system and a multi-channel pulsed high-voltage parameter-adjustable module. The intravascular tomographic imaging system may be, for example, an F series product from NANJING FORSSMANN MEDICAL TECHNOLOGY CO., LTD., for example, an intravascular tomographic imaging system (F-2), having an intravascular imaging function and a vascular calcification assessing function. The multi-channel pulsed high-voltage parameter-adjustable module has a high-voltage adjustable module, a pulse-width adjustable module, a repetition-frequency adjustable module and a pulse-burst-count adjustable module. According to the balloon imaging catheter mentioned in the first aspect of the present disclosure, balloon catheters with variable lengths and different specifications can be made, for example, balloon imaging catheters for coronary, peripheral, valvular and other parts, connected to the high-voltage adjustable module and the intravascular tomographic imaging system through the high-voltage connector and the flange connector. The multi-channel pulsed high-voltage parameter-adjustable module is a high-voltage generator module built in the intravascular tomographic imaging system, where the high-voltage generator module features adjustable high voltage, pulse width, repetition frequency and pulse burst count, is adapted to coronary, peripheral and valvular balloon imaging catheters, and provides personalized intelligent calcification treatment protocols tailored to calcifications in different parts and calcification sizes through integration with AI calcification assessment of software, thus enabling effective treatment of vascular calcifications in different parts of the human body.

The above-mentioned are merely for embodiments in the embodiments of the present disclosure. The scope of protection of the multi-channel pulsed high-voltage parameter-controllable shock wave lithotripsy balloon imaging system and the catheter thereof in the present disclosure includes, but is not limited to, the above. In the appended claims of the embodiments of the present disclosure, specific embodiments illustrated in the present disclosure can be subject to substitutions and alterations, which shall be covered within the scope of protection of the claims of the present disclosure.

What is claimed is:

1. A multi-channel pulsed high-voltage parameter-controllable shock wave lithotripsy balloon imaging system, comprising a high-voltage connector, a flange connector, a Luer taper, an intravascular tomographic imaging system, a multi-channel pulsed high-voltage parameter-adjustable module and a balloon imaging catheter, wherein the intravascular tomographic imaging system is configured to assess intravascular imaging and vascular calcification, the multi-channel pulsed high-voltage parameter-adjustable module comprises a high-voltage adjustable module, a pulse-width adjustable module, a repetition-frequency adjustable module and a pulse-burst-count adjustable module, the balloon imaging catheter is connected to the multi-channel pulsed high-voltage parameter-adjustable module via the high-voltage connector, and is connected to the intravascular tomographic imaging system via the flange connector; the Luer taper is configured to inject a shock wave transmission medium into an operation balloon, wherein the transmission medium comprises: saline and a contrast agent; the balloon imaging catheter comprises: an imaging catheter body, the operation balloon, a first operation electrode pair, a second operation electrode pair and a sheath, wherein the imaging catheter body comprises an imaging window, the operation balloon comprises an operation area, the operation balloon is provided in the imaging window, and the operation balloon is provided with a first operation balloon pin and a second operation balloon pin, wherein the first operation balloon pin and the imaging window form an enclosed space, and the second operation balloon pin and a circumferential wall of the sheath form an enclosed space; the first operation electrode pair and the second operation electrode pair are provided in the operation area of the operation balloon and on an outer wall of the imaging window;
the first operation electrode pair comprises: a first operation electrode, a second operation electrode and a first insulating layer; the second operation electrode pair comprises: a third operation electrode, a fourth operation electrode and a second insulating layer, wherein the first operation electrode is provided on the imaging window, the first insulating layer is provided on the first operation electrode, the second operation electrode is provided on the first insulating layer, the first insulating layer is provided with a first energy release window, and the second operation electrode is provided with a second energy release window, wherein the second energy release window is slightly larger than the first energy release window, and the first energy release window and the second energy release window are coaxial and co-directional; the third operation electrode is provided with a third energy release window, and the fourth operation electrode is provided with a fourth energy release window, wherein the fourth energy release window is slightly larger than the third energy release window, and the third energy release window and the fourth energy release window are coaxial and co-directional.

2. The multi-channel pulsed high-voltage parameter-controllable shock wave lithotripsy balloon imaging system according to claim 1, wherein the balloon imaging catheter comprises: a first lead wire and a second lead wire, wherein the first lead wire is connector to the first operation electrode and the third operation electrode, and the second lead wire is connected to the second operation electrode and the fourth operation electrode.

3. The multi-channel pulsed high-voltage parameter-controllable shock wave lithotripsy balloon imaging system according to claim 2, wherein the balloon imaging catheter comprises: an electrical connector, and the first lead wire and the second lead wire extend along a direction from the operation balloon to the electrical connector and are connected to the electrical connector.

4. The multi-channel pulsed high-voltage parameter-controllable shock wave lithotripsy balloon imaging system according to claim 1, wherein the balloon imaging catheter comprises: an optical lens, an optical fiber, a traction wire and an optical lens base, wherein the optical lens is connected to the optical fiber, and the traction wire is connected to the optical fiber and the optical lens base.

5. The multi-channel pulsed high-voltage parameter-controllable shock wave lithotripsy balloon imaging system according to claim 1, wherein a front end of the imaging catheter body is provided with a first rapid exchange port, and a front end of the imaging window is provided with a second rapid exchange port.

6. The multi-channel pulsed high-voltage parameter-controllable shock wave lithotripsy balloon imaging system according to claim 5, wherein the balloon imaging catheter comprises: a first developing ring, a second developing ring and a third developing ring, wherein the first developing ring is provided adjacent to the second rapid exchange port, the second developing ring and the third developing ring are provided at two ends inside the operation balloon, respectively, the second developing ring is provided adjacent to the first operation electrode pair, and the third developing ring is provided adjacent to the second operation electrode pair.

7. The multi-channel pulsed high-voltage parameter-controllable shock wave lithotripsy balloon imaging system according to claim 1, wherein the first operation electrode, the second operation electrode, the third operation electrode, and the fourth operation electrode are made of tungsten, platinum-iridium alloy or stainless steel alloy, the first operation electrode and the third operation electrode are in an annular shape, a disk shape or a square sheet shape, and the second operation electrode and the fourth operation electrode are in an annular shape, a semi-annular shape or an annular shape provided with a wire slot.
